(19) United States
(12) Patent Application Publication
(10) Pub. No.: US 2006/0036162 A1
(43) Pub. Date: Feb. 16, 2006

(54) Method and apparatus for guiding a medical instrument to a subsurface target site in a patient

(76) Inventors: Ramin Shahidi, Stanford, CA (US); Calvin R. Maurer, Mountain View, CA (US); Jay West, Mountain View, CA (US); Rasool Khadem, Louisville, CO (US)

Correspondence Address: MCANDREWS HELD & MALLOY, LTD, 500 WEST MADISON STREET, SUITE 3400, CHICAGO, IL, 60661, US

(21) Appl. No.: 11/045,013

(22) Filed: Jan. 27, 2005

Related U.S. Application Data
(60) Provisional application No. 60/541,131, filed on Feb. 2, 2004.

(51) Int. Cl. A61B 5/05

(52) U.S. Cl. 600/424

(57) ABSTRACT

Intraoperative image(s) of a patient target site are generated by an intraoperative imaging system (e.g., ultrasound or X-ray). The intraoperative imaging system is tracked with respect to the patient target site and surgical instrument(s) (e.g., a pointer, endoscope or other intraoperative video or optical device). The intraoperative images, surgical instruments, and patient target site are registered into a common coordinate system. Spatial feature(s) of the patient target site are indicated on the images of the patient target site. Indicia relating the position and orientation of the surgical instrument(s) to the spatial feature(s) of the patient target site are projected on the images, with the indicia being used to correlate the position and orientation of the surgical instruments with respect to the target feature.

### RELATED APPLICATIONS

This application makes reference to and claims priority from U.S. Provisional Patent Application Ser. No. 60/541,131 entitled “Method and Apparatus for Guiding a Medical Instrument to a Subsurface Target Site in a Patient” filed on Feb. 2, 2004, the complete subject matter of which is incorporated herein by reference in its entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

Precise imaging of portions of the anatomy is an increasingly important technique in the medical and surgical fields. In order to lessen the trauma to a patient caused by invasive surgery, techniques have been developed for performing surgical procedures within the body through small incisions with minimal invasion. These procedures generally require the surgeon to operate on portions of the anatomy that are not directly visible, or can be seen only with difficulty. Furthermore, some parts of the body contain extremely complex or small structures and it is necessary to enhance the visibility of these structures to enable the surgeon to perform more delicate procedures. In addition, planning such procedures requires the evaluation of the location and orientation of these structures within the body in order to determine the optimal surgical trajectory.

U.S. Pat. No. 6,167,296, issued Dec. 26, 2000, (Shahidi), the disclosure of which is hereby incorporated by reference in its entirety into the present application, discloses a surgical navigation system having a computer with a memory and display connected to a surgical instrument or pointer and position tracking system, so that the location and orientation of the pointer are tracked in real time and conveyed to the computer. The computer memory is loaded with data from an MRI, CT, or other volumetric scan of a patient, and this data is utilized to dynamically display 3-dimensional perspective images in real time of the patient's anatomy from the viewpoint of the pointer. The images are segmented and displayed in color to highlight selected anatomical features and to allow the viewer to see beyond obscuring surfaces and structures. The displayed image tracks the movement of the instrument during surgical procedures. The instrument may include an imaging device such as an endoscope or ultrasound transducer, and the system displays also the two images to be fused so that a combined image is displayed. The system is adapted for easy and convenient operating room use during surgical procedures.

The Shahidi 296 patent uses pre-operative volumetric scans of the patient, e.g., from an MRI, CT. Hence, it is necessary to register the preoperative volume image with the patient in the operating room. It would be beneficial to provide a navigation system that utilizes intraoperative images to eliminate the registration step. It would also be desirable to provide a system that uses intraoperative images to aid the user in navigating to a target site within the patient anatomy.

### BRIEF SUMMARY OF THE INVENTION

Certain aspects of an embodiment of the present invention relate to a system and method for aiding a user in guiding a medical instrument to a target site in a patient. The system comprises an imaging device for generating one or more intraoperative images, on which spatial features of a patient target site can be defined in a 3-dimensional coordinate system. A tracking system tracks the position and optionally, the orientation of the medical instrument and imaging device in a reference coordinate system. An indicator allows a user to indicate a spatial feature of a target site on such image(s). The system also includes a display device, an electronic computer (operably connected to said tracking system, display device, and indicator), and computer-readable code. The computer-readable code, when used to control the operation of the computer, is operable to carry out the steps of (i) recording target-site spatial information indicated by the user on said image(s), (ii) determining from the spatial feature of the target site indicated on said image(s), 3-D coordinates of the target-site spatial feature in a reference coordinate system, (iii)tracking the position of the instrument in the reference coordinate system, (iv) projecting onto a display device, a view field as seen from a known position and, optionally, a known orientation, with respect to the tool, in the reference coordinate system, and (v) projecting onto the displayed view field, indicia whose states indicate the indicated spatial feature of the target site with respect to said known position and, optionally, said known orientation. Thus, the system allows the user, by observing the states of said indicia, to guide the instrument toward the target site by moving the instrument so that said indicia are placed or held in a given state in the displayed field of view.

According to certain aspects of one embodiment of the invention, the imaging device is an x-ray (fluoroscopic) imaging device. The x-ray imaging device is capable of generating first and second digitized projection images of the patient target site from first and second positions, respectively, while the tracking device is operable to record the positions of the x-ray imaging device at the first and second positions.

According to another embodiment, the imaging device is an ultrasound imaging device and the tracking device is operable for generating tracking measurements which are recorded by the computer system when the ultrasound image(s) is generated.

The medical instrument may be any of a variety of devices, such as a pointer, a drill, or an endoscope (or other intraoperative video or optical device). When the instrument is an endoscope, the view field projected onto the display device may be the image seen by the endoscope.

A method according to certain aspects of an embodiment of the present invention involves generating one or more intraoperative images on which a spatial feature of a patient target site can be indicated, indicating a spatial feature of the target site on said image(s), using the spatial feature of the target site indicated on said image(s) to determine 3-D coordinates of the target site spatial feature in a reference coordinate system, tracking the position of the instrument in the reference coordinate system, projecting onto a display device a view field as seen from a known position and, optionally, a known orientation, with respect to the tool, in the reference coordinate system, and projecting onto the displayed view field, indicia whose states are related to the indicated spatial feature of the target site with respect to the known position and, optionally, said known orientation. This method allows the user, by observing the states of said indicia, to guide the instrument toward the target site by moving the instrument so that said indicia are placed or held in a given state in the displayed field of view.

The view field projected onto the display device may be that view as seen from the tip-end position and orientation of the medical instrument having a defined field of view. Alternatively, the view field projected onto the display device may be that a seen from a position along the axis of the instrument that is different from the tip-end . Other view fields may also be shown without departing from the scope of the present invention.

In one embodiment, the medical instrument is an endoscope. In this embodiment, the view field projected onto the display device may be the image seen by the endoscope.

The method may include the steps of generating first and second digitized projection images, such as x-ray projection images, of the patient target site from first and second positions, respectively, and indicating the spatial feature of the target site on the first and second digitized projection images.

The step of generating first and second projection images may includes moving an x-ray imaging device to a first position, to generate the first image, moving the x-ray imaging device to a second position, to generate the second image, and tracking the position of the imaging device at the first and second positions, in the reference coordinate system.

In one embodiment, target-site spatial features are indicated on the first image and then projected onto the second image. The spatial feature projected onto the second image may be used to constrain the target-site spatial feature indicated on the second image. According to one aspect of this method, the target-site spatial feature indicated on the first image is selected from an area, a line, and a point, and the corresponding spatial feature projected onto the second image is a volume, an area, and a line, respectively.

Alternatively, the indicating step may be carried out independently for both images, in which instance the 3-D coordinates of the target site are determined from the independently indicated spatial features.

According to another aspect of the present invention, the step of generating includes using an ultrasonic source to generate an ultrasonic image of the patient, and the 3-D coordinates of a spatial feature indicated on the image are determined from the 2-D coordinates of the spatial feature on the image and the position of the ultrasonic source.

In one embodiment, the target site spatial feature indicated is a volume or area, and the indicia are arranged in a geometric pattern which defines the boundary of the indicated spatial feature. According to another embodiment, the target site spatial feature indicated is a volume, area or point, and the indicia are arranged in a geometric pattern that indicates the position of a point within the target site.

According to one aspect of an embodiment of the invention, the spacing between or among indicia is indicative of the distance of the instrument from the target-site position. According to another aspect of an embodiment of the invention, the size or shape of the individual indicia is indicative of the distance of the instrument from the target-site position. According to yet another aspect of an embodiment of the invention, the size or shape of individual indicia is indicative of the orientation of said tool.

Certain embodiments of the present invention also provide the ability to define a surgical trajectory in the displayed image. Specifically, according to one embodiment, the step of indicating includes indicating on each image, a second spatial feature which, together with the first-indicated spatial feature, defines a surgical trajectory on the displayed image. According to another embodiment, the method further includes using the instrument to indicate on a patient surface region, an entry point that defines, with the indicated spatial feature, a surgical trajectory on the displayed image. In either instance, the surgical trajectory on the displayed image may be indicated by two sets of indicia, one set corresponding to the first-indicated spatial feature and the second, by the second spatial feature or entry point indicated. Alternatively, the surgical trajectory on the displayed image may for example be indicated by a geometric object defined, at its end regions, by the first-indicated spatial feature and the second spatial feature or entry point indicated.

### BRIEF DESCRIPTION OF SEVERAL VIEWS OF THE DRAWINGS

FIG. 1 is a schematic diagram of an image-guided surgery system according to certain aspects of an embodiment of the invention.

FIG. 2 is a schematic diagram depicting the architecture of a computer system which may be used in the image guided surgery system of FIG. 1.

FIG. 3 is a flow chart illustrating an image guided surgical method according to certain aspects of an embodiment of the invention.

FIG. 4 is a flow chart illustrating an image guided surgical method according to certain aspects of another embodiment of the invention.

FIG. 5 is a flow chart illustrating an image guided surgical method according to certain aspects of another embodiment of the invention.

FIG. 6 is a flow chart illustrating operating of the tracking system.

FIG. 7 is a flow chart illustrating an image guided surgical method according to certain aspects of another embodiment of the invention.

FIG. 8 is a schematic illustration of an indicating step according to one embodiment of the invention.

FIG. 9 is a schematic illustration of an indicating step according to another embodiment of the invention.

FIG. 10 illustrates a display according to an embodiment of the invention.

FIG. 11 is a schematic illustration of an indicating step according to another embodiment of the invention.

FIGS. 12-14B illustrate displays according to embodiments of the invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings

### DETAILED DESCRIPTION OF THE INVENTION

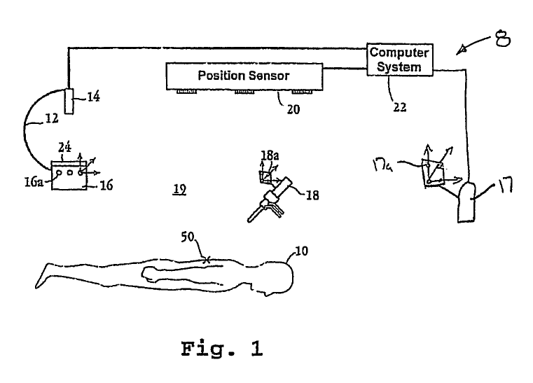

FIG. 1 is a schematic view of an image-guided surgery system 8 according to certain aspects of an embodiment of the invention. The system includes an imaging device for generating intraoperative images of selection portions of the patient's 10 anatomy. For example, as shown in FIG. 1 the imaging device may comprise a mobile fluoroscopic device 12. Fluoroscopic device 12 is preferably a C-Arm of the type which may be obtained from General Electric, Milwaukee, Wis. The mobile fluoroscopic device includes an X-ray camera 14 and an image intensifier 16. Alternatively, the imaging device may be an ultrasound imaging device, such as a hand held ultrasound imaging probe 17. The system also includes a surgical instrument 18, which may be any of a variety of devices such as a pointer, a drill, or an endoscope, for example. The system also includes a tracking system. In this respect, the C-arm/image intensifier 24, the ultrasound probe 17 and the surgical instrument 18 are each equipped with tracking elements 16a, 17a and 18a, respectively, that define local coordinate systems for each of those components. In the illustrated embodiment, the tracking elements 16a, 17a, 18a are emitters, such as infrared light-emitting diode (LED) markers. The tracking elements communicate with a position sensor (e.g. a camera (digitizer)) 20, such as an Optotrak digitizer available from Northern Digital, Waterloo, Ontario, Canada. While an active, optical tracking system is shown in the illustrated embodiment, it will be appreciated that other tracking systems may alternatively be used. For example, the optical system may employ passive tracking elements, e.g. reflectors. Alternatively, an electromagnetic (EM) tracking system or a combined EM/optical tracking system may be employed.

The position sensor 20 tracks the components 12, 17, 18 within an operating space 19, and supplies data needed to perform coordinate transformations between the various local coordinate systems to a computer system 22, such as a workstation computer of the type available from Sun Microsystems, Mountain View, Calif. Or Silicon Graphics Inc., Mountain View, Calif. The NTSC video output of camera 14 is also processed by the computer system. A video framegrabber board, such as an SLIC-Video available from Osprey Systems, Cary, N.C., may also be employed to allow loading of gray-scale images from the video buffer of the C-arm to the computer system.

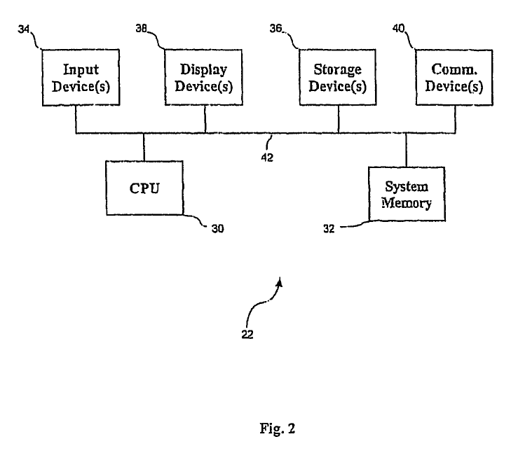

The general architecture of such a computer system 22 is shown in more detail in FIG. 2. The computer system includes a central processing unit (CPU) 30 that provides computing resources and controls the computer. CPU 30 may be implemented with a microprocessor or the like, and may also include a graphics processor and/or a floating point coprocessor for mathematical computations. Computer 22 also includes system memory 32 which may be in the form of random-access memory (RAM) and random-access memory (ROM). Input device(s) 34, such as a keyboard, mouse, foot pedal, stylus, etc., are used to input data into the computer. Storage device(s) 36 include a storage medium such as magnetic tape or disk, or optical disk, e.g., a compact disk, that are used to record programs of instructions for operating systems, utilities and applications. The storage device(s) may be internal, such as a hard disk and may also include a disk drive for reading data and software embodied on external storage mediums such as compact disks, etc. Storage device 36 may be used to store one or more programs and data that implement various aspects of the present invention, including the imaging and tracking procedures. One or more display devices 38 are used to display various images to the surgeon during the surgical procedure. Display device(s) 38 are preferably high-resolution device(s). The computer system may also include communications device(s) 40, such as a modem or other network device for making connection to a network, such as a local area network (LAN), Internet, etc. With such an arrangement, program(s) and/or data that implement various aspects of the present invention may be transmitted to computer 22 from a remote location (e.g., a server or another workstation) over a network. All major system components of the computer may connect to a bus 42 which may be more than one physical bus. Bus 42 is preferably a high-bandwidth bus to improve speed of image display during the procedure.

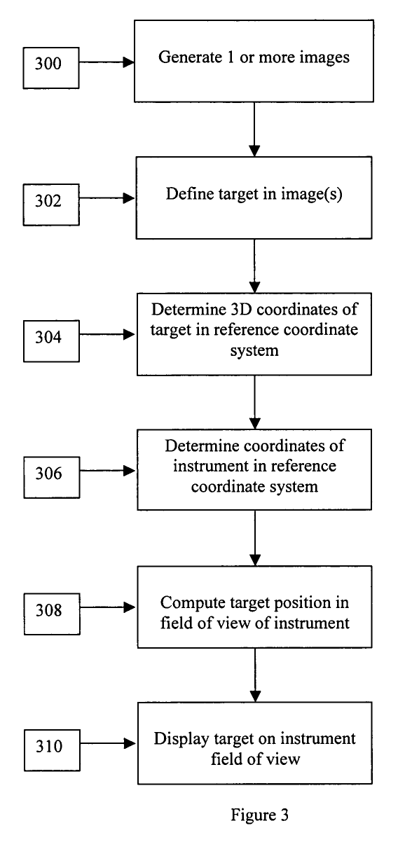

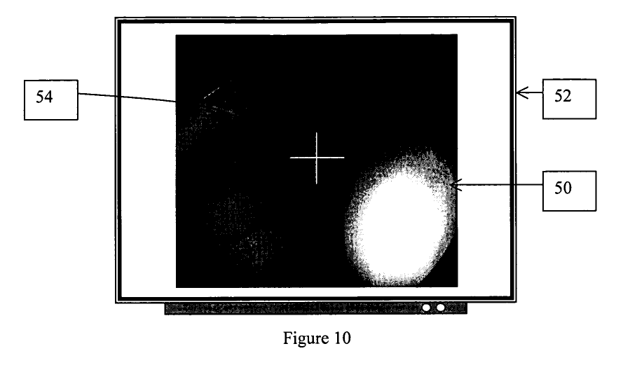

FIG. 3 is a flow chart illustrating an image guided surgical method according to certain aspects of an embodiment of the invention. Initially, in step 300 an imaging device, such as the fluoroscopic device 12 or the ultrasound probe 17, is used to generate at least one image of the patient 10. The image(s) is/are transmitted to the computer 22, e.g., by means of a cable connecting the imaging device to the computer, and by means of the video capture device installed in the computer. Next, in step 302 the user defines the target in the image(s). This step may be accomplished, for example, by moving the cursor to the desired image position(s) and double-clicking the mouse. Next in step 304, the 3-D coordinates of the target are determined in the reference coordinate system. In particular, the coordinates of the selected target in the reference coordinate system are computed using the tracking measurements recorded when the image(s) was/were generated. As will be appreciated, in the context of X-ray images the tracking elements 16a are positioned to allow parameters of the fluoroscopic device 12, such as focal length and image center, to be estimated. Next, in step 306 the coordinates of the instrument 18 are determined in the reference coordinate system. Specifically, using tracking measurements recorded by the tracking system, the position and orientation of the instrument 18 in the reference coordinate system are computed by the computer system 22. Next, in step 308 the computer system computes the target position in the field of view of the instrument 18. Specifically, using the now known transformation between reference and instrument coordinate systems, the coordinates of the selected target in the instrument coordinate system are computed. Next, in step 310 the computer displays the coordinates of the target on the instrument's field of view. For example, as is illustrated in FIG. 10, the instrument 18 may be an endoscope, in which case the field of view projected onto the display device may be the image as seen by the endoscope. For an instrument, such as an endoscope, with a defined field of view the view field projected onto the display device can be that view seen from the tip-end position and orientation of the medical instrument. Alternatively, the view field projected onto the display device can be that view seen from a position along the axis of the instrument that is different from the tip-end position of the medical instrument. For example, where the instrument is a pointer, the user can select the view field from a position, e.g., distal from the tip of the pointer, along the axis of the pointer.

In FIG. 10, the real-time image 50 from the endoscope is displayed on a monitor 52. An indicia, illustrated as a cross hair 54, is projected onto the displayed field of view of the endoscope. As the endoscope moves relative to the target site, the cross hair moves to guide the user towards the target site. In particular, when the endoscope is centered on the target, the cross hair 54 will be centered on the image 50. Hence the cross hair 54 functions as an indicia whose state (position in this instance) is related to the indicated spatial feature target site with the known position of the endoscope. As a result, the user, by observing the state (position) of the cross hair, can guide the endoscope toward the target site by moving the endoscope so that the cross hair is placed in the center of the display.

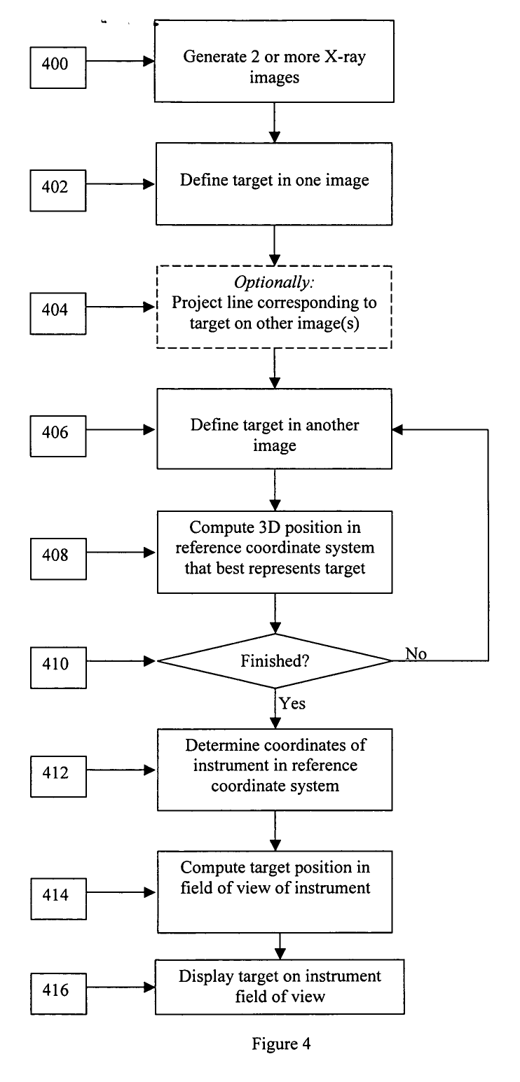

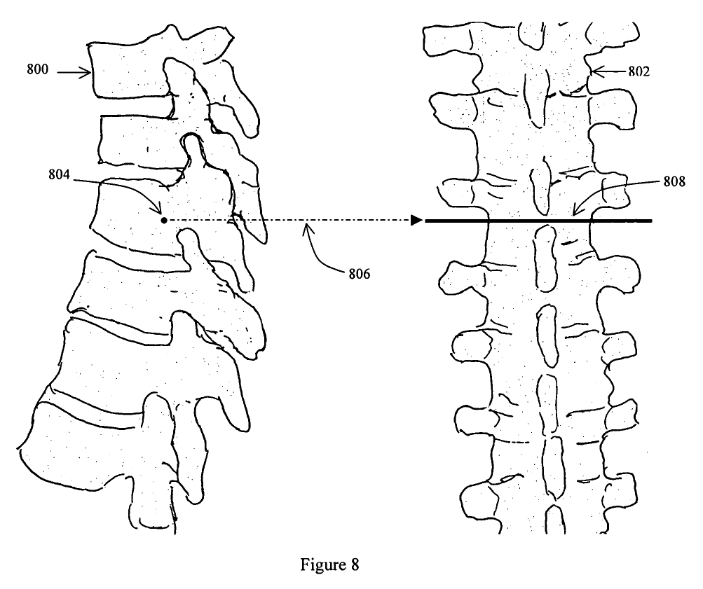

FIG. 4 is a flow chart illustrating an image guided surgical method according to certain aspects of another embodiment of the invention. In step 400, the fluoroscopic device 12 is used to generate two or more X-ray images of the patient. For example, as is shown in FIG. 8, the fluoroscopic device can be used to make first and second images 800, 802 of the patient target site taken from first and second positions, respectively. In the illustrated embodiment, the patient target site is a portion of the spine and the first image 800 is a lateral view of the spine portion, while the second image 802 is an anterior-posterior (AP) view of the spine portion. The first and second images 800, 802 are generated by moving the fluoroscopic device 12 to a first position to generate the first image, moving the fluoroscopic device to a second position to generate a second image, and tracking the position of the imaging device, i.e., with the tracking system, at the first and second positions in the reference coordinate system.

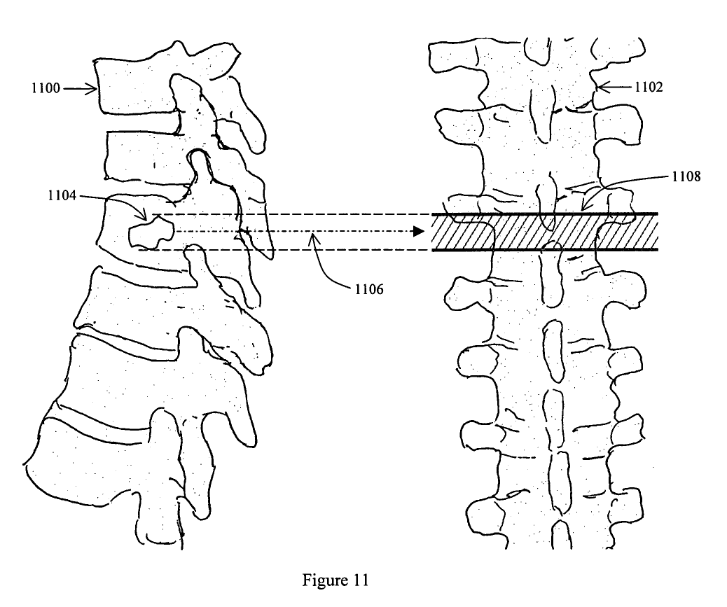

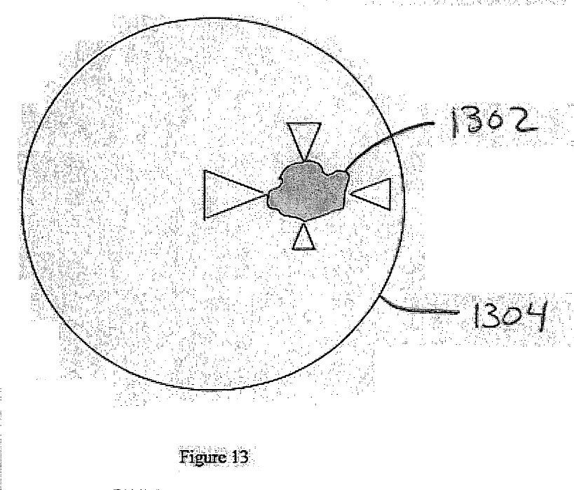

Referring again to FIG. 4, the X-ray images are transmitted to the computer system 22, e.g., by means of a cable connecting the fluoroscopic device to the computer system, and by means of a video capture device installed in the computer system. In step 402 the user selects the desired position of the target in one image. This step may be accomplished, for example, by moving the cursor to the desired image position and double-clicking the computer mouse. Because fluoroscopic images are projective, a point selected in one image corresponds to a line in space in the other images. As an optional step, the computer system may draw the line representing the target on the other X-ray image(s). For example, referring to FIG. 8, the user initially selects a target 804 in the first image 800. The computer system projects 806 the point 804 onto the second image 802 as a line 808. In FIG. 8, the target-site spatial feature indicated on the first image is shown as a point and the corresponding spatial feature projected onto the second images is a line. Alternatively, the target-site spatial feature on the first image can be selected as area or a line, in which case the corresponding spatial feature projected onto the second image is a volume or an area, respectively. Where the target site spatial feature indicated is a volume or area, a geometric pattern, which defines the boundary of the indicated special feature, may be projected onto the second image. For example, FIG. 11 shows first and second images 1100, 1102. The target-site spatial feature indicated in the first image 1100 is an area 1104 that is projected 1106 onto the second image 1102 as a geometric pattern 1108. Where the target site spatial feature indicated is a volume or area, the indicia can be arranged in a geometric pattern which defines the boundary of the indicated spatial feature in the image that is displayed to the user during navigation. (See, e.g., FIG. 13 where geometric shape 1302 is displayed over the instrument's field of view 1304). Alternatively, wherein the target site spatial feature is indicated as a volume, area or point, the displayed indicia can be arranged in a geometric pattern that indicates the position of a point within the target site.

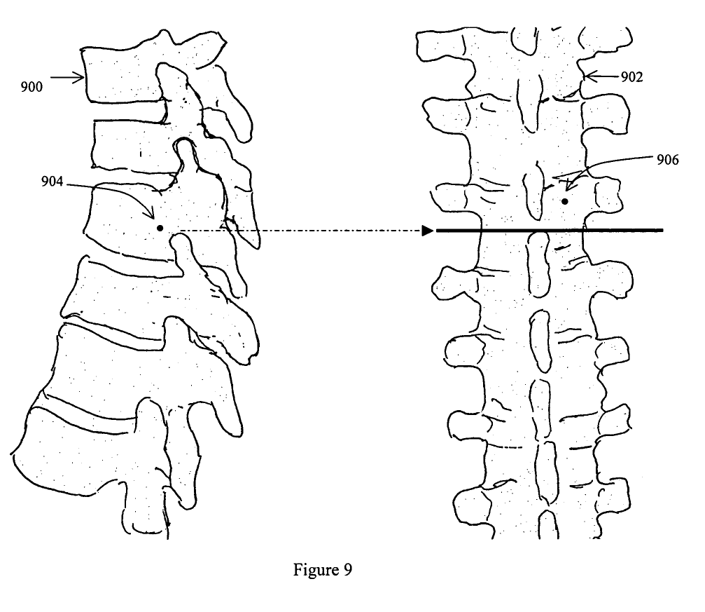

Referring again to FIG. 4, in step 406 the user defines a target in another image by moving the cursor to the desired position in that image and double-clicking the mouse. The line 808 projected in the second image 802 can function as a guide for directing the user to the target area in the second image that aligns with the target area selected in the first image. Optionally, the projected special feature, e.g., the line 808, can be used to constrain where the target-site spatial feature can be indicated on the second image. Specifically, in some applications it may be desirable to only allow the user to select a point on the line 808 when defining the target in the second image (and any further images). Alternatively, in some applications it may be desirable to perform the indicating step independently for each image. In such instances it may still be desirable to project a line into the other image(s) to aid the user in selecting the target in the other image(s). This is illustrated generally in FIG. 9, which shows first and second images 900, 902. As can be seen, the target (point) 904 selected in the first image 900 does not align with the target (point) 906 selected in the second image 902.

After the target is selected in the second image, the coordinates of the point best representing the selected target in the reference coordinate system are computed using the tracking measurements recorded when the X-ray image(s) were generated (step 408). Steps 406 through 410 can be repeated to allow the user to define the target in additional images. When more than two images are used, step 408 can be accomplished, for example, by using a matrix that is minimized to give the best match of all of the points selected in the images. Once the user is finished defining the target in the images, control is passed to step 412 where coordinates of the instrument 18 are determined in the reference coordinate system. Specifically, using tracking measurements recorded by the tracking system, the position and orientation of the instrument 18 in the reference coordinate system are computed by the computer system 22. Next, in step 414 the computer system computes the target position in the field of view of the instrument 18. Specifically, using the now known transformation between reference and instrument coordinate systems, the coordinates of the selected target in the instrument coordinate system are computed. Next, in step 416 the computer displays the coordinates of the target on the instrument's field of view.

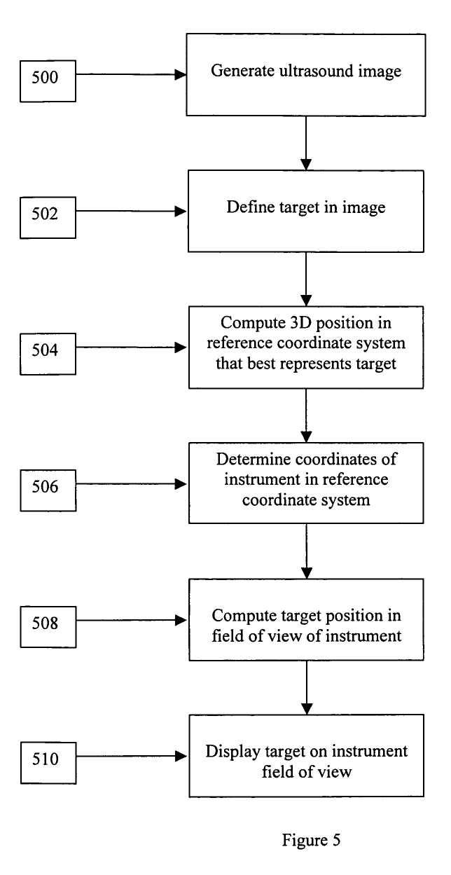

FIG. 5 is a flow chart illustrating an image guided surgical method according to certain aspects of another embodiment of the invention. In this embodiment, an ultrasound scanner 17 is used to generate the intraoperative image. In step 502 the user generates an ultrasound image of the patient using an ultrasound scanner 17 in the OR. The ultrasound image is transmitted to the computer system 22, e.g., by means of a video cable connecting the ultrasound scanner to the computer system and by means of a video capture device installed in the computer system. Next, in step 504 the user selects the target position in the ultrasound image, e.g., by moving the cursor to the desired location and double-clicking the mouse. Next, in step 506 the 3D coordinates of the target are determined in the reference coordinate system. Specifically, the tracking system installed in the OR is used to track the position of ultrasound scanner during the imaging process. The computer system uses the tracking measurements recorded when the ultrasound image was generated to compute the point best representing the selected target in the reference coordinate system. Next, in step 506 the coordinates of the instrument 18 are determined in the reference coordinate system. Specifically, using tracking measurements recorded by the tracking system, the position and orientation of the instrument 18 in the reference coordinate system are computed by the computer system 22. Next, in step 508 the computer system computes the target position in the field of view of the instrument 18. Specifically, using the now known transformation between reference and instrument coordinate systems, the coordinates of the selected target in the instrument coordinate system are computed. Next, in step 510 the computer displays the coordinates of the target on the instrument's field of view.

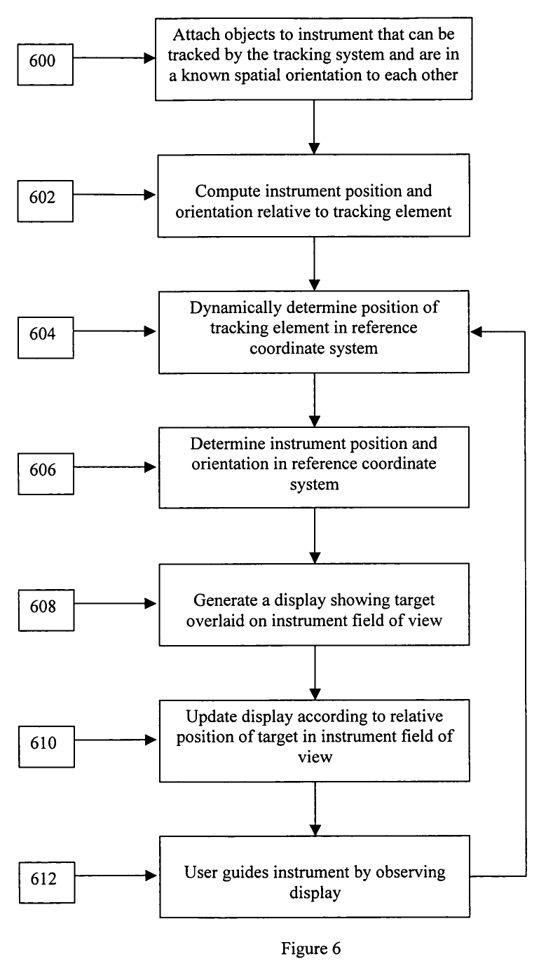

FIG. 6 is a flow chart that further illustrates how the navigation system is used to guide the instrument during a procedure. In step 600, the instrument is equipped with a tracking element 18a so the instrument can be tracked by the position sensor 20. In step 602 the instrument's position and orientation with respect to the tracking elements 18a are computed. In step 604 the current position of the tracking element 18a in the reference coordinate system is measured by means of the position sensor 20. Using the known transformation between the instrument coordinate system and that of the tracking element 18a, the position and orientation of the instrument in the reference coordinate system are computed in step 606. The position of the target in the instrument coordinate system is computed, using the known transformation between instrument and reference coordinates. In step 608, the computer system 22 generates a display showing the target overlaid on the instrument's field of view. The display is updated according to the relative position of the target in the instrument's field of view in step 610. In step 612, the user guides the instrument by observing the display and moving or rotating the instrument to achieve a desired position of the target in the instrument's field of view. Steps 604 through 612 are continuously repeated to update the display as the user moves the instrument.

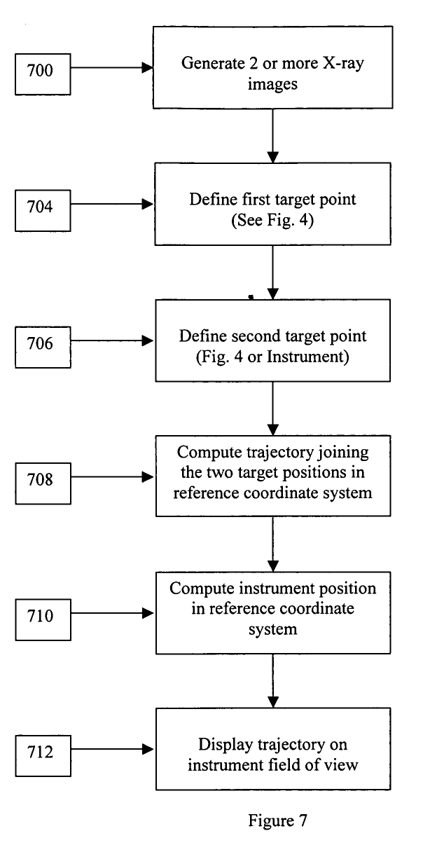

FIG. 7 is a flow chart illustrating an image guided surgical method according to certain aspects of another embodiment of the invention. This embodiment provides the ability to define a surgical trajectory in the displayed image. Initially, in step 700 the fluoroscopic device 12 is used to generate two or more X-ray images of the patient 10. The images are transmitted to the computer system 22, for example by means of a cable connecting the fluoroscopic device to the computer system and by means of a video capture device installed in the computer system. In step 704 a first target point is defined in the reference coordinate by selecting its position in two or more images. The target-defining step 704 can be accomplished in the manner described above in connection with FIG. 4. Next, in step 706 a second target point is defined in the reference coordinate system by selecting its position in two or more images in the manner shown in FIG. 4. Alternatively, the instrument 18 can be used to indicate on a patient surface region, an entry point that defines the second target point. The trajectory including the two target points in the reference coordinate system is calculated in step 708. Next, in step 710 the coordinates of the instrument 18 are determined in the reference coordinate system. Specifically, using tracking measurements recorded by the tracking system, the position and orientation of the instrument 18 in the reference coordinate system are computed by the computer system 22. Using the known transformation between instrument and reference coordinates, the computer displays the trajectory including the two target points on the instrument's field of view 712. The surgical trajectory on the displayed image may, for example, be indicated by two sets of indicia, one set corresponding to the first-indicated spatial feature and the second corresponding to either the second indicated spatial feature or indicated entry point. Alternatively, the surgical trajectory on the displayed image may, for example, be indicated by a geometric object defined, at its end regions, by the first-indicated spatial feature and the second spatial feature or entry point indicated.

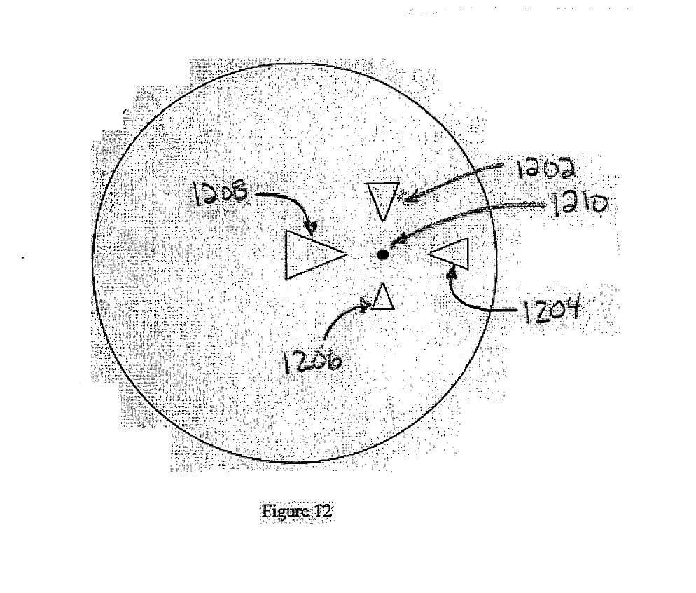

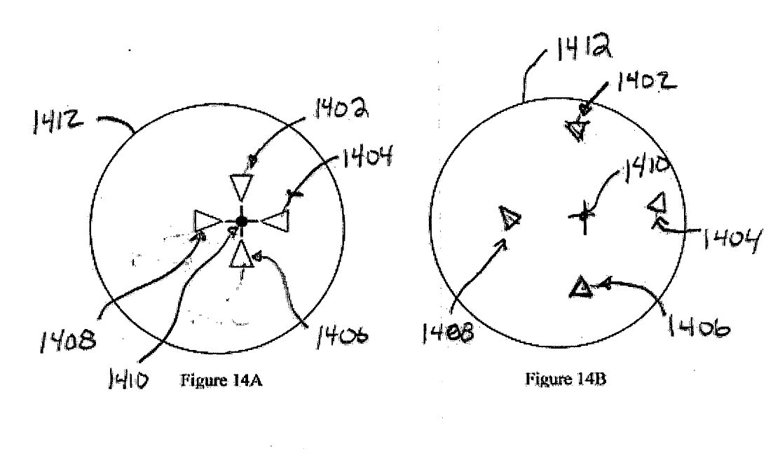

A variety of display methods can be used to guide the user during navigation. For example, the size or shape of the individual indicia may be used to indicate the orientation of the instrument relative to the target-site. This is illustrated in FIG. 12, where the indicia are displayed as four arrows 1202-1208 and a point 1210 is used to represent the target. As the instrument 18 moves relative to the target site in the patient, the sizes of the arrows 1202-1208. For example, a larger arrow, such as the down arrow 1202, indicates that the instrument needs to be moved down relative to the target. Similarly, the larger size of the right pointing arrow 1208 relative to the left pointing arrow 1204 indicates that the instrument needs to be moved to the right. Alternatively or additionally, the display can be structured such that the size or shape of individual indicia indicates the distance of the instrument from the target site. For example, the size of the arrows could increase or decrease to indicate the relative distance from the target. In such a display, the location of the target on displayed field of view could be indicative of the relative alignment of the instrument with the target. Specifically, the instrument is aligned with the target when the displayed target, e.g., point 1210, is centered in the displayed field of view. Alternatively or additionally, the spacing between or among indicia may be used to indicative of the distance of the instrument from the target-site position. This is illustrated in FIG. 14A and 14B. In this example, the indicia are displayed as four arrows 1402-1408. As the instrument 18 moves closer to the target site in the patient, the arrows 1402-1408 move farther from the display target 1410. Hence, the relative spacing of the arrows 1402-1408 from the target 1410 is used to show indicate the relative distance form the target while the location of the target on displayed field of view 1412 is indicative of the relative alignment of the instrument with the target. As will be appreciated, a variety of other display methods can be employed without departing from the scope of the present invention.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for assisting a user in guiding a medical instrument to a subsurface target site in a patient, comprising:
generating one or more intraoperative images on which a spatial feature of a patient target site can be indicated;
indicating a spatial feature of the target site on said image(s);
using the spatial feature of the target site indicated on said image(s) to determine 3-D coordinates of the target site spatial feature in a reference coordinate system;
tracking the position of the instrument in the reference coordinate system;
projecting onto a display device, a view field as seen from a known position and, optionally, a known orientation, with respect to the instrument, in the reference coordinate system; and
projecting onto the displayed view field, indicia whose states are related to the indicated spatial feature of the target site with respect to said known position and, optionally, said known orientation;
whereby the user, by observing the states of said indicia, can guide the instrument toward the target site by moving the instrument so that said indicia are placed or held in a given state in the displayed field of view.

2. The method of claim 1, wherein said generating and indicating include the steps of
generating first and second digitized projection images of the patient target site from first and second positions, respectively; and
indicating the spatial feature of the target site on the first and second digitized projection images.

3. The method of claim 2, wherein said projection images are x-ray projection images.

4. The method of claim 2, which further includes, after indicating the spatial feature of the target site on the first image, projecting the target-site spatial feature indicated in the first image onto the second image, and using the spatial feature projected onto the second image to constrain the target-site spatial feature indicated on the second image.

5. The method of claim 4, wherein the target-site spatial feature indicated on the first image is selected from an area, a line, and a point, and the corresponding spatial feature projected onto the second image is a volume, an area, and a line, respectively.

6. The method of claim 2, wherein said indicating is carried out independently for both images, and the 3-D coordinates of the target site are determined from the independently indicated spatial features.

7. The method of claim 2 wherein said generating includes moving an x-ray imaging device to a first position, to generate said first image, moving the x-ray imaging device to a second position, to generate said second image, and tracking the position of the imaging device at said first and second positions, in said reference coordinate system.

8. The method of claim 1, wherein said generating includes using an ultrasonic source to generate an ultrasonic image of the patient, and the 3-D coordinates of a spatial feature indicated on said image are determined from the 2-D coordinates of the spatial feature on the image and the position of the ultrasonic source.

9. The method of claim 1, wherein said medical instrument is an endoscope and the view field projected onto the display device is the image seen by the endoscope.

10. The method of claim 1, wherein the view field projected onto the display device is that seen from the tip-end position and orientation of the medical instrument having a defined field of view.

11. The method of claim 1, wherein the view field projected onto the display device is that seen from a position along the axis of instrument that is different than the tip-end position of the medical instrument.

12. The method of claim 1, wherein the target site spatial feature indicated is a volume or area, and said indicia are arranged in a geometric pattern which defines the boundary of the indicated spatial feature.

13. The method of claim 1, wherein the target site spatial feature indicated is a volume, area or point, and said indicia are arranged in a geometric pattern that indicates the position of a point within the target site.

14. The method of claim 1, wherein the spacing between or among indicia is indicative of the distance of the instrument from the target-site position.

15. The method of claim 1, wherein the size or shape of the individual indicia is indicative of the distance of the instrument from the target-site position.

16. The method of claim 1, wherein the size or shape of individual indicia is indicative of the orientation of said instrument.

17. The method of claim 1, wherein said indicating includes indicating on each image, a second spatial feature which, together with the first-indicated spatial feature, defines a surgical trajectory on the displayed image.

18. The method of claim 1, which further includes using said instrument to indicate on a patient surface region, an entry point that defines, with said indicated spatial feature, a surgical trajectory on the displayed image.

19. The method of claims 17 or 18, wherein the surgical trajectory on the displayed image is indicated by two sets of indicia, one set corresponding to the first-indicated spatial feature and the second, by the second spatial feature or entry point indicated.

20. The method of claims 17 or 18, wherein the surgical trajectory on the displayed image is indicated by a geometric object defined, at its end regions, by the first-indicated spatial feature and the second spatial feature or entry point indicated.

21. A system designed to a user in guiding a medical instrument to a target site in a patient, comprising:
(a) an imaging device for generating one or more intraoperative images, on which spatial features of a patient target site can be defined in a 3-dimensional coordinate system;
(b) a tracking system for tracking the position and optionally, the orientation of the medical instrument and imaging device in a reference coordinate system;
(c) an indicator by which a user can indicate a spatial feature of a target site on such image(s);
(d) a display device;
(e) an electronic computer operably connected to said tracking system, display device, and indicator, and
(f) computer-readable code which is operable, when used to control the operation of the computer, to carry out the steps of:
(i) recording target-site spatial information indicated by the user on said image(s), through the use of said indicator,
(ii) determining from the spatial feature of the target site indicated on said image(s), 3-D coordinates of the target-site spatial feature in a reference coordinate system,
(iii) tracking the position of the instrument in the reference coordinate system,
(iv) projecting onto a display device, a view field as seen from a known position and, optionally, a known orientation, with respect to the instrument, in the reference coordinate system, and
(v) projecting onto the displayed view field, indicia whose states indicate the indicated spatial feature of the target site with respect to said known position and, optionally, said known orientation;
whereby the user, by observing the states of said indicia, can guide the instrument toward the target site by moving the instrument so that said indicia are placed or held in a given state in the displayed field of view.

22. The system of claim 21, wherein said imaging device is an x-ray imaging device capable of generating first and second digitized projection images of the patient target site from first and second positions, respectively, and said tracking device is operable to record the positions of the imaging device at said two positions.

23. The system of claim 21, wherein said medical instrument is an endoscope and the view field projected onto the display device is the image seen by the endoscope.

24. Machine readable code in a system designed to assist a user in guiding a medical instrument to a target site in a patient, said system including:
(a) an imaging device for generating one or more intraoperative images, on which a patient target site can be defined in a 3-dimensional coordinate system;
(b) a tracking system for tracking the position and optionally, the orientation of the medical instrument and imaging device in a reference coordinate system;
(c) an indicator by which a user can indicate a spatial feature of a target site on such image(s);
(d) a display device, and (e) an electronic computer operably connected to said tracking system, display device, and indicator; and
said code being operable, when used to control the operation of said computer, to
(i) recording target-site spatial information indicated by the user on said image(s), through the use of said indicator,
(ii) determining from the spatial feature of the target site indicated on said image(s), 3-D coordinates of the target-site spatial feature in a reference coordinate system,
(iii) tracking the position of the instrument in the reference coordinate system,
(iv) projecting onto a display device, a view field as seen from a known position and, optionally, a known orientation, with respect to the instrument, in the reference coordinate system, and
(v) projecting onto the displayed view field, indicia whose states indicate the indicated spatial feature of the target site with respect to said known position and, optionally, said known orientation;
whereby the user, by observing the states of said indicia, can guide the instrument toward the target site by moving the instrument so that said indicia are placed or held in a given state in the displayed field of view.
